(19) United States
(12) Patent Application Publication
(10) Pub. No.: US 2026/0090551 A1
(43) Pub. Date: Apr. 2, 2026

(54) OLEOSOME COMPOSITION

(71) Applicant: CARGILL, INCORPORATED, Wayzata, MN (US)

(72) Inventors: Daniel Pierre Anna ABTS, Lier (BE); Nils BILLECKE, Sint-Genesius-Rode (BE); Eliane Yvonne GOOSSENS, NIEL (BE)

(73) Assignee: CARGILL, INCORPORATED, Wayzata, MN (US)

(21) Appl. No.: 19/113,580

(22) PCT Filed: Sep. 27, 2023
(86) PCT No.: PCT/US2023/075196

§ 371 Date: Mar. 20, 2025

(30) Foreign Application Priority Data
Sep. 28, 2022 (EP) 22198403.2

(51) Int. Cl. A23B 2/758; A23L 33/00; A61K 8/02; A61K 8/37; A61K 9/107; A61K 31/23; A61Q 19/00

(52) U.S. Cl. CPC A23B 2/758; A23L 33/40; A61K 8/0291; A61K 8/37; A61K 9/1075; A61K 31/23; A61Q 19/00; A61K 2800/10

(57) ABSTRACT

Present invention relates to an oleosome composition comprising loaded isolated vegetable oleosomes being isolated vegetable oleosomes loaded with one or more sources of triglycerides having saturated C16 fatty acids that are positioned at the sn2 position, which are present in the inside of the loaded oleosomes and wherein at least 80 wt. % of total weight of lipids in composition is present in the oleosomes. It further relates to the use of isolated vegetable oleosomes as a carrier for these triglycerides. Present invention moreover relates to a product comprising the oleosome composition, wherein product is selected from the group consisting of food products, feed products, pharmaceutical products, personal care products, nutritional compositions, and industrial products. Present invention further relates to a process for loading isolated vegetable oleosomes.

### CROSS-REFERENCE TO RELATED APPLICATIONS

This application claims the benefit of European Application No. 22198403.2, filed Sep. 28, 2022, which is incorporated by reference herein in its entirety.

### FIELD OF THE INVENTION

The present invention relates to an oleosome composition comprising loaded isolated oleosomes being isolated oleosomes loaded with one or more sources of triglycerides having saturated C16 fatty acids that are positioned at the sn2 position and which are present in the inside of said loaded isolated oleosomes. The present invention further relates to a product comprising the oleosome composition according to the present invention. The present invention also relates to a process for loading isolated oleosomes with one or more sources of triglycerides having saturated C16 fatty acids that are positioned at the sn2 position to prepare an oleosome composition according to the present invention and to the use of the oleosome composition.

### BACKGROUND OF THE INVENTION

Oleosomes, also known as “oil bodies”, “lipid bodies”, “lipid droplets” or “spherosomes”, are pre-emulsified droplets or vesicles of oil stored in plant seeds and used as energy sources for plant growth and metabolism. Oleosomes are typically extracted from cells by a process of soaking, washing and grinding the seeds in the presence of water and subsequently filtering or decanting to remove solids and form an aqueous suspension. The suspension is centrifuged to separate the oleosomes, called isolated oleosomes. The size of the isolated oleosomes may further be enlarged. WO2021126408A1 by the present applicant provides a process for enlarging oleosomes.

Lipophilic bioactive compounds such as lipids, vitamins, and phytochemicals serve important antioxidant, functional, nutritional, and structural roles in the human/animal body. A substantial number of these bioactive compounds are highly lipophilic such as polyunsaturated lipids.

Existing colloidal systems may be suitable matrices for the protection and delivery of these compounds. However, there is still a need for an improved matrix for delivering triglycerides having saturated C16 fatty acids that are positioned at the sn2 position that is providing improved oxidative stability and having improved stability in the gastric phase of the human digestive tract. There is a need for a carrier providing oxidative stability while allowing the tailoring of the fatty acid profile according to the desired need. There is a need for such carrier which can be incorporated in nutritional compositions. The present invention addresses this need.

### STATEMENT OF THE INVENTION

The invention relates to an oleosome composition comprising loaded isolated vegetable oleosomes being isolated vegetable oleosomes loaded with one or more sources of triglycerides having saturated C16 fatty acids that are positioned at the sn2 position, which are present in the inside of said loaded isolated vegetable oleosomes and wherein at least 80 wt. % of the total weight of lipids in the composition is present in the oleosomes.

Furthermore, the invention relates to a product comprising the oleosome composition, wherein said product is selected from the group consisting of food products, feed products, pharmaceutical products, personal care products, nutritional compositions and industrial products.

The present invention also relates to a process for preparing the oleosomes composition and the process comprises the steps of:
a) blending one or more sources of triglycerides having saturated C16 fatty acids that are positioned at the sn2 position and optionally other lipids with isolated vegetable oleosomes in a ratio of the source of triglycerides having saturated C16 fatty acids that are positioned at the sn2 position and optionally other lipids to oleosomes of from 1:99 to 95:5; and
b) subjecting the blend obtained from step a) to a high-shear force and obtaining an oleosome composition comprising loaded isolated vegetable oleosomes being isolated vegetable oleosomes loaded with one or more sources of triglycerides having saturated C16 fatty acids that are positioned at the sn2 position, which are present at the inside of said loaded isolated vegetable oleosomes and wherein at least 80 wt. % of the total weight of lipids in the composition is present in the oleosomes.

Finally, the invention relates to a use of the oleosome composition as a carrier for triglycerides having saturated C16 fatty acids that are positioned at the sn2 position. More specifically, the invention relates to a use of the oleosome composition for the prevention of oxidation of the triglycerides having saturated C16 fatty acids that are positioned at the sn2 position and/or improving the stability of the triglycerides having saturated C16 fatty acids that are positioned at the sn2 position in the gastric phase of the human digestive tract.

Below several preferred features are disclosed. These features are applicable to the oleosome composition as well as to the product comprising the oleosome composition, the use of the oleosome composition and the process for loading isolated vegetable oleosomes with one or more sources of triglycerides having saturated C16 fatty acids that are positioned at the sn2 position to prepare the oleosome composition.

### DETAILED DESCRIPTION

The present invention is elucidated below with a detailed description. When used in this specification and claims, the terms “comprises” and “comprising” and variations thereof mean that the specified features, steps or integers are included. The terms are not to be interpreted to exclude the presence of other features, steps or components.

### Oleosome Composition

The present invention is related to an oleosome composition (or oleosomes composition) comprising loaded isolated vegetable oleosomes being isolated vegetable oleosomes loaded with one or more sources of triglycerides having saturated C16 fatty acids that are positioned at the sn2 position which are present in the inside of said loaded isolated vegetable oleosomes and wherein at least 80 wt. % of the total weight of lipids in the composition is present in the oleosomes. Preferably, at least 90 wt. % or at least 95 wt. % or at least 98 wt. % of the total weight of lipids in the composition is present in the oleosomes. In fact, from 96 wt. % to 99.8 wt. %, from 97 wt. % to 99.0 wt. % of the total weight of lipids in the composition is present in the oleosomes.

“Total weight of lipids” as used in the present description means the weight of all the lipids present in the composition, thus both present in the oleosomes (in the center as well as in the interphase layer) and outside of the oleosomes, being the free lipids. Preferably, substantially all the lipids are present in the oleosomes. In the current invention, the term “lipid” or “lipids” is encompassing free fatty acids, mono-, di-, tri-glycerides, and phospholipids.

The amount of free lipids in the oleosome composition can be quantified by extracting the free lipids from the oleosome composition with heptane. The heptane will only extract the free lipids, not the lipids inside the oleosomes. Subsequently the amount of lipids in the heptane phase is quantified. Quantification can be done by means of GPC analysis.

The amount of lipids in the oleosome composition that is present in the oleosomes is calculated as the difference between the total amount of lipids (for example measured by Soxhlet method) and the amount of free lipids in the oleosome composition.

Per definition, oleosomes comprise lipids being phospholipids (and optionally diacylglycerides (DAGs), monoacylglycerides (MAGs), free fatty acids (FFAs), and one or more combinations thereof) at the interphase as well as triglycerides (TAGs) in the center of the oleosomes. Oleosomes are present as vesicles of oil storage in their natural source. Once isolated/removed from their natural source, they are known as isolated oleosomes. By loading of oleosomes, additional lipids (in the present invention a source of triglycerides having saturated C16 fatty acids that are positioned at the sn2 position) are added into the oleosomes according to the present invention. Moreover, lipids that may be present in the composition and that are not inside the oleosomes (so-called “free lipids”), are limited.

### Isolated Vegetable Oleosomes

“Isolated vegetable oleosomes” as used in the present description, means oleosomes that have been isolated/removed from their natural source, i.e. vegetable source. The term encompasses oleosomes isolated from a single oleosome source, i.e. single strain or seed line of vegetable seeds. The term “isolated vegetable oleosomes” also encompasses blends of oleosomes that are isolated from more than one oleosome source. i.e. multiple strains and/or seed lines of vegetable seeds or combination of seeds of different vegetables sources. These isolated oleosomes may be present in the composition according to the invention and still be called “isolated”.

Oleosomes are comprising intrinsic proteins such as mainly oleosin and minor amounts of caleosin and steroleosin. Without wishing to be bound by a particular theory, the present inventors observed that the oleosins contain a hydrophilic part, which is present at the isolated oleosomes' surface, and a hydrophobic part which is anchored in the oil in the center of the oleosomes and ensures for oleosome stability. Even at alkaline conditions of pH 8 or higher, intrinsic proteins remain strongly bound, whereas weakly bound proteins will be removed in alkaline conditions.

The isolated oleosomes may have a content of intrinsic proteins in an amount of from 0.2 to 10 wt. % based on the dry weight of the isolated oleosomes, such as from 0.3 to 8 wt. %, or from 0.3 to 6 wt. %. The applied method is described in the experimental section in paragraphs [0117]-[0120] of WO2021126408A1 which section is incorporated by reference. The isolated oleosomes may have a content of phospholipids in a range of from 0.2 to 6.0 wt. %, from 0.3 to 5.5 wt. %, from 0.4 to 5.0 wt. % based on the dry weight of the isolated oleosomes. The content of phospholipids may be determined using total phosphorus methods (AOCS Ca 19-86, AOCS Ca 12-55), TLC followed by GC. HPLC, or 31P-NMR. “Dry weight of the isolated oleosomes” as used in the present description means the weight of the dry (non-aqueous) part of the isolated oleosomes. The isolated oleosomes obtained by the extraction/isolation process are usually in the form of a creamy paste comprising a certain amount of water.

The present invention differs from artificial oleosomes or artificial oil bodies (AOBs) in which a free oil e.g., fish oil is mixed with phospholipids and oleosin protein to form AOBs (=synthetic emulsions). The present invention comprises loaded isolated natural oleosomes that are loaded with lipids and the lipids are present in the inside of said loaded isolated vegetable oleosomes. The present composition is free of AOBs.

### Vegetable Source

The vegetable source for obtaining isolated vegetable oleosomes may be cells from pollens, spores, seeds or vegetative plant organs in which oleosomes or oleosomes-like organelles are present, preferably a plant seed.

The vegetable source may be a member of the Brassicaceae, Amaranthaceae, Asparagaceae, Echium, Glycine, Astaraceae, Fabaceae, Malvaceae, Faboidae, Aracaceae, Euphorbiceae, Sinapsis, Lamiaceae, Cyperaceae, Anacardiaceae, Rosaceae, Betulaceae, Juglandaceae, Oleaceae, Lauraceae, Sapotaceae and/or Poaceae families.

Preferably, the vegetable source is a plant seed from the group of plant species comprising: rapeseed (Brassica spp.), soy bean (Glycine max), sunflower (Helianthus annuits)—and their corresponding mid or high oleic varieties—, cottonseed (Gossypium spp.), coconut (Cocus nucifera), linseed/flax (Linum usitatissimum) (including brown (also called bronze) and yellow (also called gold) linseed, hazelnut (Corylus avellana), maize and maize germ (Zea mays), almond (Prunus dulcis), cashew (Anacardium occidentale), olive (Olea), avocado (Persea americana), and shea (Butyrospermum parkii). Other examples that may be used are oil of palm (Elaeis guineeis), groundnut (Arachis hypogaea), castor (Ricinus communis), safflower (Carthamus tinctorius), mustard (Brassica spp. and Sinapis alba), coriander (Coriandrum sativum), squash (Cucurbita maxima), Brazil nut (Bertholletia excelsa), walnut (Juglands major), jojoba (Simmondsia chinensis), thale cress (Arabidopsis thaliana), wheat and wheat germ (Triticum spp.), amaranth (family of Amaranthus), sesame (Sesamum indicum), oat (Avena sativa), camelina (Camelina sativa), lupin (Lupinus), peanut (Arachis hypogaea), quinoa (Chenopodium quinoa), chia (Salvia hispanica), yucca, cocoa bean (Theobroma cacao), argan (Argania spinosa), and rice. For all of these, any variety with increased level of unsaturated fatty acids compared to the original seed variety may be used. Varieties may be obtained by natural selection or by genetic modification (GMO).

Preferably, the isolated vegetable oleosomes are sourced from a vegetable source selected from the group consisting of rapeseed, soybean, cottonseed, coconut, brown linseed, yellow linseed, hazelnut, maize, sesame, almond, cashew, olive, avocado, shea, and sunflower, and their corresponding mid or high oleic varieties, and any variety with increased level of unsaturated fatty acids compared to the original variety.

The isolated vegetable oleosomes in the oleosome composition according to the present invention may be selected from the group consisting of isolated rapeseed oleosomes, isolated soybean oleosomes, isolated sunflower oleosomes, isolated linseed oleosomes, and a combination of two or more thereof.

The isolated linseed oleosomes may be isolated yellow linseed oleosomes and/or isolated brown linseed oleosomes.

The isolated sunflower oleosomes may be isolated mid-oleic (MO) sunflower oleosomes, isolated high-oleic (HO) sunflower oleosomes, high oleic-high stearic (HOHS) sunflower seeds. HP sunflower seeds (HP), high oleic-high palmitic (HOHP) sunflower seeds, or any combination of two or more thereof.

More preferably, the isolated vegetable oleosomes are sourced from a vegetable source selected from the group consisting of rapeseed and rapeseed varieties with increased level of unsaturated fatty acids compared to the original rapeseed, sunflower, mid and high oleic sunflower, soybean, coconut, brown linseed, yellow linseed and hazelnut. Most preferably, the isolated vegetable oleosomes are sourced from a vegetable source selected from the group consisting of rapeseed, sunflower, mid and high oleic sunflower, soybean, brown linseed and yellow linseed.

“Sunflower” as used in the present description means any type of sunflower seed belonging to the species Helianthus annuus. Several types of sunflower seeds exist, each characterized by the composition of the fatty acid profile of the oil present in these seeds. Regular sunflower seeds contain sunflower oil that is characterized by a typical composition of 66 to 72 wt. % linoleic acid (LA), 9 to 13 wt. % saturated acids, such as palmitic acid (PA) and stearic acid (SA), 17 to 23 wt. % oleic acid, and less than 1 wt. % of ALA, expressed on the total weight of fatty acid moiety of the oil. Other well-known varieties of sunflower seeds are so-called mid-oleic (MO) sunflower seeds, high-oleic (HO) sunflower seeds, high oleic-high stearic (HOHS) sunflower seeds, high-palmitic sunflower seeds (HP) and high oleic-high palmitic (HOHP) sunflower seeds, which can be obtained by natural selection or by genetic modification (GMO). Typically, high-oleic sunflower oil is characterized by a content of 7 to 11 wt. % LA, 7 to 11 wt. % saturated acids (PA and SA), 80 to 86 wt. % oleic acid, and less than 1 wt. % ALA, all expressed on the total weight of fatty acid moiety of the oil. Typically, mid-oleic sunflower oil is characterized by a content of 24 to 28 wt. % LA, 7 to 11 wt. % saturated acids (PA and SA), 62 to 68 wt. % oleic acid, and less than 1 wt. % ALA, all expressed on the total weight of fatty acid moiety of the oil.

“Rapeseed” as used in the present description means any type of rapeseed belonging to the species Brassica napus. Typically rapeseed oil (low erucic) has a fatty acid profile comprising palmitic acid (PA) (C16:0) in an amount of 4 wt. %, stearic acid (SA) (C18:0) in an amount of 2 wt. %, oleic acid (OA) (C18:1) in an amount of 56 wt. %, linoleic acid (LA) (C18:2) in an amount of 26 wt. %, and linolenic acid (ALA) (C18:3) in an amount of 10 wt. %, expressed on the total weight of fatty acid moiety of the oil (see Bailey's Industrial Oil and Fat Products, 6th edition, 6th volume, 2005, Chapter 6 vegetable oils, Table 2). Rapeseed oil lends itself to genetic modification, and several rapeseed varieties giving oils with modified fatty acid profile have been developed. “Isolated rapeseed oleosomes” as used in the present description means oleosomes that have been isolated/removed from rapeseed.

“Soy bean” as used in the present description means any type of soy bean belonging to the species Glycine max. Raw soy beans contain approx. 18 wt. % of soy bean oil that has a fatty acid profile comprising palmitic acid (C16:0) in an amount of 11 wt. % (range 7-14 wt. %), stearic acid (C18:0) in an amount of 4 wt. %, oleic acid (C18:1) in an amount of 22 wt. % (range 19-30 wt. %), linoleic acid (C18:2) in an amount of 53 wt. % (range 44-62 wt. %), and linolenic acid (ALA) (C18:3) in an amount of 8 wt. % (range 4-11 wt. %), expressed on the total weight of fatty acid moiety of the oil (see Bailey's Industrial Oil and Fat Products, 6th edition, 6th volume, 2005.

Chapter 6 vegetable oils, Table 2 and section 5.15). “Isolated soy bean oleosomes” as used in the present description means oleosomes that have been isolated/removed from soy beans.

“Linseed” as used in the present description means any type of linseed belonging to the species Linum usitatissimum. Linseed produces a vegetable oil that is highly unsaturated and that is known as linseed oil. Regular linseed oil (brown linseed) has a fatty acid profile comprising palmitic acid (C16:0) in an amount of 6 wt. %, stearic acid (C18:0) in an amount of 3 wt. %, oleic acid (C18:1) in an amount of 17 wt. %, linoleic acid (C18:2) in an amount of 14 wt. %, and linolenic acid (C18:3) in an amount of 60 wt. %, expressed on the total weight of fatty acid moiety of the oil (see Bailey's Industrial Oil and Fat Products, 6th edition, 6th volume, 2005, Chapter 6 vegetable oils. Table 2). There is a different type of engineered linseed (yellow) having a low level of linolenic acid (2%) and a high level of linoleic acid. “Isolated linseed oleosomes” as used in the present description means oleosomes that have been isolated/removed from linseeds.

The term “fatty acid profile” of a substance, such as an oil, a fat, isolated oleosomes, or an oleosome composition, as used in the present description, mean the total of fatty acids that is present in the oily substance in the form of free fatty acids and in the form of the fatty acid moiety of a lipid (monoglyceride, diglyceride or triglyceride). For example, if an oil is comprising an amount of oleic acid expressed on total weight of the fatty acid profile, this amount is the total of oleic acid present in the oil as a free fatty acid and as oleic acid bound that is bound as the fatty acid moiety in the triglycerides, diglycerides and monoglycerides that are present in the oil.

Isolated vegetable oleosomes can also be obtained from algal origin. In an aspect of the invention, oleosomes sourced from algae are excluded from and do not fall within the scope of the present invention.

### Loaded Isolated Vegetable Oleosomes

The present invention is related to compositions comprising loaded isolated oleosomes being isolated vegetable oleosomes loaded with one or more sources of triglycerides having saturated C16 fatty acids that are positioned at the sn2 position and which are present in the inside of said oleosomes.

“Loaded isolated (vegetable) oleosomes,” “isolated (vegetable) oleosomes loaded with . . . ” as used in the present description means that additional components, such as lipids, are encapsulated by the (vegetable) oleosomes, thus present in the inside thereof.

As stated, it is preferred that a large amount (at least 80 wt. %), substantially all, or all of the lipids is present in loaded isolated vegetable oleosomes. The remaining amount of lipids. i.e. the amount of lipids that is not present in the loaded isolated vegetable oleosomes expressed on the total amount of lipids that are present in the oleosome composition, may be present in the composition in free form, e.g., outside of the oleosomes; the composition then comprises free lipids in addition to loaded isolated oleosomes. In an aspect, substantially all lipids are present inside oleosomes for optimal protection of these lipids. The combination of lipids that are present in the oleosomes and the free lipids thus adds up to 100 wt. % based on the total weight of lipids in the oleosome composition.

“Triglyceride having saturated C16 fatty acids that are positioned at the sn2 position” as used in the present description means a triglyceride having at the 2-position of its glycerol backbone (sn2 position) a saturated C16 fatty acid. Examples thereof are the following triglycerides: 1,3-dioleoyl-2-palmitoyl glyceride (OPO), 1,3-dilinoleoyl-2-palmitoyl glyceride (LPL), and 1-oleoyl-2-palmitoyl-3-linoleoyl glyceride (OPL), wherein the palmitic acid (saturated C16) is present at the 2-position. “O” stands for oleic acid, “P” for palmitic acid, and “L” for linoleic acid. OPO is known to be an important component of human breast milk.

The one or more sources of triglycerides having saturated C16 fatty acids that are positioned at the sn2 position may have a fatty acid profile comprising from 30 to 50% by weight of saturated fatty acids having 16 carbon atoms and from 30 to 70% by weight of unsaturated fatty acids having 18 carbon atoms based on the total weight of the fatty acid profile of the one or more source, wherein 50 to 80%, such as from 52 to 75% by weight of the saturated fatty acids having 16 carbon atoms are present at the 2-position of a triglyceride (the remaining part being present at the 1,3 positions).

The oleosome composition according to the invention may comprise the one or more sources of triglycerides having saturated C16 fatty acids that are positioned at the sn2 position in an amount of from 0.5 to 90.0 wt. %, preferably from 1.0 to 80.0 wt. %, more preferably from 1.5 to 70 wt. % based on the total dry weight of the oleosome composition.

The oleosome composition according to the invention may comprise an amount of saturated C16 fatty acids that is positioned at the sn2 position of a triglyceride is in a range of from 0.075 to 36.0 wt. %, such as from 0.1 to 36.0 wt. %, preferably from 0.15 to 32 wt. %, such as 0.2 to 32.0 wt. %, more preferably 0.5 to 28.0 wt. % or from 0.234 to 26.25 wt. %, based on the total weight of the fatty acid profile of the oleosome composition.

Depending on the desired use of the oleosome compositions, the ranges may vary. For example, for a use in which nearly all fat in the composition is to be added in the form of oleosomes, e.g. for an infant formula, the ranges saturated C16 fatty acids that is positioned at the sn2 position of a triglyceride can be from 5.0 to 25.0 wt. %, from 8.0 to 20.0 wt. %, or from 10.0 to 18.0 wt. % based on the total weight of the fatty acid profile of the oleosome composition. For example, for a use in which in addition to oleosomes also free fats will be used, oleosomes with a higher range of saturated C16 fatty acids that is positioned at the sn2 position of a triglyceride may be used (e.g. between 18.0 wt. % and 25 wt. %) and for oleosomes that will be used without addition of free fat, oleosomes with a lower range of saturated C16 fatty acids that is positioned at the sn2 position of a triglyceride may be used (e.g. between 5.0 wt. % and 12.0 wt. %). Moreover, for even more concentrated requirements, the oleosome composition may comprise saturated C16 fatty acids that are positioned at the sn2 position of a triglyceride in an amount of from 10.0 to 50.0 wt. %, 16.0 to 40 wt. %, 20.0 to 36.0 wt. % based on the total weight of the fatty acid profile of the oleosome composition.

### Other Lipids

The total amount of lipids in the oleosome composition may be up to 99.8 wt. % based on the total dry weight of the oleosome composition. For example, the total amount of lipids in the oleosome composition may be between 80.0 and 99.6 wt. %, from 85.0 to 99.0 wt. %, or from 90.0 to 98.0 wt. % based on the total dry weight of the oleosome composition. For example, the total amount of lipids in the oleosome composition may be between 80.0 and 99.7 wt. %, from 85.0 to 99.0 wt. %, or from 90.0 to 99.0 wt. % based on the total dry weight of the oleosome composition. The majority of the remaining part of the dry matter of the oleosome composition are the proteins.

In addition to loading the isolated vegetable oleosomes with one or more sources of triglycerides having saturated C16 fatty acids that are positioned at the sn2 position to obtain loaded isolated vegetable oleosomes comprising one or more sources of triglycerides having saturated C16 fatty acids that are positioned at the sn2 position, one or more other lipids may be loaded into the isolated vegetable oleosomes. Examples thereof are ALA. LA, LC-PUFA, MCFA or combinations of two or more thereof.

“ALA” as used in the present description means alpha-linolenic acid or α-linolenic acid, which is an omega-3 fatty acid, and its shorthand name is 18:3 (ω-3) or 18:3 (n-3). In the present description, ALA is referring only to C18:3 ccc wherein ccc stands for three cis double bonds.

As a source of ALA, flaxseed oil may be used. In an example, flaxseed oil is comprising ALA in an amount of from 55 to 60 wt. %, preferably from 57 to 59 wt. %, such as 58 wt. % based on total weight of the fatty acid profile of the oil.

“LA” as used in the present description means linoleic acid, which is an omega-6 fatty acid, and its shorthand name is 18:2 (ω-6) or 18:2 (n-6).

“LC-PUFA” as used in the present description means long-chain poly-unsaturated fatty acids. These poly-unsaturated fatty acids have a chain (“backbone”) comprising 20 or more carbon atoms and comprise more than one double bond in their backbone.

Examples of specific LC-PUFA fatty acids are the following:
ARA (or AA) or arachidonic acid, which is an omega-6 fatty acid, and its shorthand name is 20:4 (ω-6) or 20:4 (n-6) or 20:4n6
DHA or docosahexaenoic acid, which is an omega-3 fatty acid, and its shorthand name is 22:6 (ω-3) or 22:6 (n-3) or 22:6n3
EPA or eicosapentaenoic acid or icosapentaenoic acid, which is an omega-3 fatty acid, and its shorthand name is 20:5 (ω-3) or 20:5 (n-3) or 20:5n3
DPA or docosapentaenoic acid, which is an omega-3 fatty acid, and its shorthand name is 22:5 (ω-3) or 22:5 (n-3).

The LC-PUFA may be selected from the group consisting of arachidonic acid (ARA), docosahexaenoic acid (DHA), eicosapentaenoic acid (EPA), docosapentaenoic acid (DPA), and combinations of two or more thereof. As a source of LC-PUFA a source of DHA and/or a source of ARA may be used. As a source of DHA Schizochytrium (a unicellular coastal marine eukaryote) oil may be used. As a source of ARA Mortierella alpina (a soil fungus) oil may be used. In an example, Schizochytrium oil is comprising DHA (being C22:6n3) in an amount of from 37 to 50 wt. %, preferably from 44 to 48 wt. %, for example 46 wt. % based on total weight of the fatty acid profile of the oil. In an example, Mortierella alpina oil is comprising ARA (being C20:4n6) in an amount of from 40 to 50 wt. %, preferably from 44 to 48 wt. %, for example 46 wt. % based on total weight of the fatty acid profile of the oil.

“MCFA” or “medium-chain fatty acids” as used in the present description means saturated fatty acids with a carbon chain length of 6, 8, 10, or 12 carbon atoms, MCFA are naturally found in a variety of lipid sources including coconut oil, palm kernel oil, dairy lipids, or fractions thereof, such as C6 and C8 rich fractions of coconut oil.

In the oleosome composition, the fatty acid profile of the lipids may have a ratio of omega-6 fatty acids over omega-3 fatty acids (n-6/n-3 or ω-6/ω-3 or n-6:n-3 or ω-6:ω-3) in a range of from 0.2:1 to 36.0:1, more preferably from 0.7:1 to 12:1, more preferably from 1.4:1 to 5.0:1, more preferably from 0.7:1 to 11.5:1.

The oleosomes may be further loaded with one or more source of ALA and/or long-chain polyunsaturated fatty acids (LC-PUFA). The oleosome composition may comprise a combined amount of ALA and/or LC-PUFA of from 1.0 to 7.0 wt. %, from 1.5 to 6.0 wt. %, or from 2.0 to 5.0 wt. % based on the total weight of the fatty acid profile of the oleosome composition. The minimum amount of LC-PUFA and/or ALA may be regulated for specific products, e.g., according to EU regulations for infant formula. The maximum amount may also be regulated for specific products. The amount of LC-PUFA in the oleosome composition may be at least 0.5 wt. %, 1.0 wt. %, 2.0 wt. % based on the total weight of the fatty acid profile of the oleosome composition. More specifically, the amount of DHA in the oleosome composition may be in a range of from 0.33 to 1.14 wt. % based on the total weight of the fatty acid profile of the oleosome composition. The amount of ALA may be at least 0.3 wt. %, at least 0.5 wt. %, or at least 1.0 wt. % based on the total weight of the fatty acid profile of the oleosome composition.

The amount of DHA in the oleosome composition may be in a range of from 0.33 to 1.14 weight % based on the total weight of lipids, especially for infant food products.

### Lipophilic Dietary Bioactive Substances

In one aspect of the invention, the oleosome composition may comprise one or more sources of one or more lipophilic dietary bioactive substance, selected from the group consisting of oil-soluble vitamins, phytosterols, curcuminoids, carotenoids, and flavonoids. More preferably, the oleosome composition may comprise one or more lipophilic dietary bioactive substance, selected from the group consisting of oil-soluble vitamins, phytosterols, carotenoids, and flavonoids. Most preferably, the oleosome composition may comprise one or more lipophilic dietary bioactive substance, selected from the group consisting of oil-soluble vitamins and phytosterols.

The term “lipophilic dietary bioactive substances” as used in the current description encompasses the bioactive substance as such, as well as its form esterified to a fatty acid. Esterification may make dietary bioactive substances more fat-soluble (i.e.; lipophilic) so they may be easily loaded into the isolated oleosomes.

Lipophilic dietary bioactive substances are dietary bioactive substances that are lipophilic (non-polar) in nature. Dietary bioactive substances or dietary bioactives are terms commonly used to describe food components which, although they are not essential, may exert a positive effect on one or more physiological processes and hence may be beneficial to health. Their non-polar nature is often a limiting factor for their incorporation into commercial food products due to incompatibility with many food matrices. Furthermore, many hydrophobic bioactive components in foods are sensitive to food processing and storage and are poorly bioaccessible. Examples of oil-soluble vitamins are vitamin D, vitamin E, vitamin A, vitamin K, and ascorbyl palmitate (a derivative of vitamin C). The lipophilic dietary bioactive substances according to the present invention do not include the lipids ALA, LC-PUFA, and the other lipids specified above. Examples of phytosterols are plant sterols, such as β-sitosterol, campesterol, and stigmasterol, and plant stanols, such as sitostanol and campestanol. In a preferred aspect of the invention, the oleosome composition may comprise one or more lipophilic dietary bioactive substance, selected from the group consisting of vitamin D, vitamin E, vitamin A, vitamin K, ascorbyl palmitate, β-sitosterol, campesterol, stigmasterol, sitostanol, campestanol, and their fatty acid-bound esters.

Each of the one or more lipophilic dietary bioactive substance may be present in the oleosome composition in an amount of from 0.1 to 1000 microgram per gram of the total amount of lipids, from 0.2 to 800 microgram per gram of the total amount of lipids, or from 0.3 to 600 microgram per gram, of the total amount of lipids. More specifically, the oleosome composition according to the invention is comprising lipophilic dietary bioactive substances in an amount of from 0.1 to 200 microgram per gram, such as 0.3 microgram per gram, 144 microgram per gram, based on the total weight of lipids of the oleosome composition, preferably from 50 to 900, from 100 to 800, from 200 to 700, or at least 500 microgram per gram of lipids.

In a specific aspect of the oleosome composition, vitamin D may be present as a lipophilic dietary bioactive substance; preferably wherein the content of vitamin D is in a range of from 0.1 to 1000 microgram, from 0.2 to 800 microgram, or from 0.3 to 600 microgram per gram, based on the total weight of lipids of the oleosome composition. Depending on the use of the oleosome composition, vitamin D may be present in an amount that is at the lower end of the range, e.g., for a nutritional composition given the EU regulation recommending a daily intake for infants of 10 micrograms per day and adults 15 micrograms per day. Vitamin D may also be present in an amount of the upper end of the range above, e.g., when used as “fortification” or supplements.

### Additional Ingredients

Other ingredients may be added to the composition. e.g., to stabilize the oleosomes in the composition. Examples thereof are proteins or thickeners, the latter leading to an increase in viscosity which in turn may lead to more stable compositions.

### Loaded Isolated Vegetable Oleosome Size and Form

The average globule diameter of the loaded isolated vegetable oleosomes is expressed as the D50-value. The D50-value of oleosomes is the diameter below which 50% of the volume of oleosome particles lies, and it is expressed in micron (=micrometer, symbol: μm). To measure the average globule diameter (D50-value) of the oleosomes, the oleosomes are considered spherical and in the case of non-spherical oleosomes, the diameter is considered as being the largest dimension that can be measured between two opposite points on the surface thereof.

To be able to measure the globule diameter of the oleosomes, a Mastersizer 3000 from Malvern equipped with a Hydro module was used during the measurements. A refractive index of 1.47 is used to measure the oleosomes size. The concentration of the oleosomes in the buffer is such that an obscuration in the range from 8.0 to 8.5% in the Mastersizer equipment will be obtained. Obscuration within the Mastersizer is the amount of light blocked or scattered, by the particles. Therefore, the oleosomes are diluted in a buffer solution containing 10 mM sodium phosphate. pH 7.4, and 1.0 wt. % sodium dodecyl sulfate (SDS). For example, about 0.2 wt. % of oleosomes is diluted in the buffer solution and the dilution is further adjusted to obtain the obscuration. Once this optimal obscuration is obtained, the globule diameter is measured, and the average globule diameter (D50-value) can be calculated.

The loaded isolated vegetable oleosomes in the oleosome composition may have an average globule diameter of the oleosomes in a range of from 0.3 to 15 microns, preferably from 0.6 to 8.0 microns, more preferably from 1.0 to 5.5 microns.

Typically, loaded isolated vegetable oleosomes have an average globule diameter of about 0.3 to 2.5 microns, from 0.6 to 2.2 microns, or from 1.0 to 2.0 microns.

Preferably, the loaded isolated oleosomes in the oleosome composition have an average globule diameter in a range of from more than 2.5 to 15 microns, preferably from 3.0 to 12 microns, more preferably from 4.0 to 10 microns. Such a size is important when being used for infant formula since the average globule size of human breast milk is about 5 microns. For obtaining the loaded isolated oleosomes having an average globule diameter in a range of from 2.5 to 15 microns an additional process step of enlarging the isolated oleosomes prior to, subsequently to or simultaneously to loading, may be required.

The oleosome composition of the present invention may be in paste form or powder form. The powder form may be obtained after the isolated oleosomes have been loaded. e.g., by spray drying, fluid bed drying, freeze-drying, or vacuum drying. A carrier such as for example maltodextrin may be added to the composition prior to spray-drying. The obtained powder may be used as a supplement.

### Product Comprising the Oleosome Composition

The invention also relates to products comprising the oleosome composition of the present invention, wherein the product is selected from the group consisting of food products, feed products, pharmaceutical products, personal care products, nutritional compositions, and industrial products.

The product comprising the oleosome composition according to the invention, may be a nutritional composition comprising the oleosome composition in a range of from 0.1 to 70.0 wt. %, preferably between 2.0 and 50.0 wt. % on dry matter of the nutritional composition. For example, in an amount of from 5.0 to 65.0 wt. %, from 9.3 to 60.0 wt. %, or from 20.6 to 55.0 wt. % on dry matter of the nutritional composition. More specifically in an amount of from 0.2 to 22.0 wt. %, such as 0.3 wt. %, 3 wt. %, 10 wt. %, 21 wt. %. The nutritional composition is further comprising at least one additional nutritional ingredient, other than oleosomes. The at least one nutritional ingredient is not derived from oleosomes. Nutritional ingredients are ingredients that contribute to the caloric intake and/or provide micronutrients. With “on dry matter of the nutritional composition” is meant that it is expressed on all components except water.

The additional nutritional ingredient may be selected from the group consisting of sources of proteins, sources of fats, sources of carbohydrates, sources of micronutrients, and combinations of two or more thereof. These sources do not comprise oleosomes. Preferably, these sources do not comprise isolated vegetable oleosomes, isolated oleosomes from any other origin, loaded isolated oleosomes from any other origin or a combination of two or more thereof.

The term “micronutrients” is encompassing nutrients that an organism needs in small quantities for the proper functioning of its metabolism. Examples of micronutrients are, but are not limited to vitamins, minerals, trace elements, essential amino acids and essential fatty acids.

The additional nutritional ingredient may, more preferably, be selected from the group consisting of proteins, carbohydrates, fats, minerals, trace elements, essential amino acids, essential fatty acids, vitamins, and a combination of two or more thereof.

When sources of fats (lipids) are added to the product according to the invention, these fats are free fats that are added after the preparation of the loaded isolated oleosomes. These are hence not present inside the loaded isolated oleosomes. The fatty acid profile of the oleosome composition will hence not necessarily be the fatty acid profile of the final product.

The nutritional composition may further comprises one or more non-nutritional ingredient. Non-nutritional ingredients according to the invention are ingredients that do not substantially add to the caloric intake and/or do not substantially provide micronutrients. Examples of non-nutritional ingredients are flavors, colorants, emulsifiers, acid regulators such as citric acid or lactic acid, preservatives, and the like. The non-nutritional ingredients may be from a natural or synthetic origin.

In the nutritional composition the combination of the oleosome composition, the at least one additional nutritional ingredient and optionally water and/or non-nutritional ingredients make up 100 wt. %.

Examples of nutritional compositions according to the present invention may be compositions that are developed to cover the nutritional needs, either as a supplement or as complete nutrition. The people that are targeted for the nutritional composition according to the invention relate to specific groups of people, such as, but not limited to, preterm infants, infants, toddlers, elderly people, pregnant women, athletes, or humans having nutritional deficiencies and/or having a deficient immune system. In a specific aspect, the nutritional composition is an infant formula. This infant formula may be milk-based, e.g. with milk protein isolate and/or caseinate and/or whey protein, or it may be plant-based, e.g. with almond, soy, rice, corn and/or pea proteins. In a specific aspect, the nutritional composition is for pregnant women. The nutritional compositions may be designed for people suffering from a more specific disease state such as cancer, chronic obstructive pulmonary disease, and later-stage kidney disease, and others. Amongst others, nutritional compositions may be helpful for people who struggle with a loss of appetite, have difficulty with chewing, have trouble preparing balanced meals, and/or are recovering from surgery or an illness. If the nutritional composition is meant for complete nutrition, it can provide a healthy balance of protein, carbohydrate, and/or fat.

These nutritional compositions can be in the form of liquid, as a ready-to-drink formula, or used in feeding tubes. It can also be in the form of a formula base i.e., a powder or a concentrated liquid, to be dissolved in water or another fluid for the preparation of a ready-to-drink nutritional composition. The nutritional composition may also be in the form of a pudding or a jelly, or the form of a cookie or a snack bar, or any other form.

In yet another aspect of the invention, the at least one nutritional ingredient other than oleosomes is not an emulsifier.

In one specific aspect, the nutritional composition according to the invention comprising oleosome composition is a food product, such as infant formula, preferably growing-up milk. The infant food product or infant formula is a term well-known in the art and it refers to food that is specifically manufactured for infants and it may be characterized in that is soft, and easily consumable by infants and has a nutritional composition adapted to the specific needs at each growth stage.

The infant food product according to the invention may be in the form of a liquid, as a ready-to-drink infant food product. It can also be in the form of a formula base, i.e., a powder or a concentrated liquid, to be dissolved in water or another fluid for the preparation of a ready-to-drink infant food product. The infant food product may also be in the form of a pudding or a jelly, or in the form of a cookie or a snack bar, or in any other form. The infant food product of the present invention is encompassing the three forms available on the market, i.e., powder (infant base powder), liquid concentrate, and ready-to-feed liquids.

It may be a first age infant formula, for infants from birth to age of 6 months, a follow-on formula (also called second age infant formula), for infants from an age of 6 to 12 months, or a growing-up formula (also called third age infant formula) for infants from the age 1 to 3 years. Preferably, the infant food product of the present invention is a first age infant formula.

In another specific aspect, the nutritional composition according to the invention comprising the oleosome composition is a nutritional composition, such as a nutritional drink, a nutritional powder, such as a sport nutrition powder; a powder for food fortification, a nutritional bar or cookie, such as a sports nutrition bar; or a nutritional supplement.

In one aspect, the nutritional composition is an infant formula, preferably a growing-up milk, and the infant formula
has a total content of lipids in a range of from 2.5 to 4.5 wt. %, from 2.8 to 4.0 wt. %, or from 3.0 to 3.4 wt. % on total weight of the infant formula,
wherein at least 90%, at least 95%, or even substantially all of the lipids of the infant formula is present in the oleosome composition, and
   wherein the oleosome composition
is comprising loaded isolated vegetable oleosomes being isolated vegetable oleosomes loaded with one or more sources of triglycerides having saturated C16 fatty acids that are positioned at the sn2 position; which are present in the inside of said loaded isolated vegetable oleosomes, and
is comprising an amount of triglycerides having saturated C16 fatty acids that are positioned at the sn2 position of a triglyceride in a range of from 5.0 to 25.0 wt. %, from 8.0 to 20.0 wt. %, or from 10.0 to 18.0 wt. % based on the total weight of the fatty acid profile of the oleosome composition, and
wherein at least 80 wt. % of the total weight of lipids in the oleosome composition is present in the oleosomes.

Examples of other types of food and feed products include but are not limited to drinks such as coffee, black tea, powdered green tea, cocoa, and juice; milk component-containing drinks, such as raw milk, processed milk, and lactic acid drinks; a variety of drinks including nutrition-enriched drinks, such as calcium-fortified drinks and the like and dietary fiber-containing drinks; dairy products, such as butter, cheese, vegan cheese, yoghurt, coffee whitener, whipping cream, custard cream, and custard pudding; iced products such as ice cream, soft cream, lacto-ice, ice milk, sherbet, and frozen yogurt; processed fat food products, such as mayonnaise, margarine, spread, and shortening; soups; stews; seasonings such as sauce and dressings; a variety of paste condiments represented by kneaded mustard; a variety of fillings typified by jam and flour paste; a variety or gel or paste-like food products including red bean-jam, jelly, and foods for swallowing impaired people; food products containing cereals as the main component, such as bread, noodles, pasta, pizza pie, and corn flake; Japanese, US and European cakes, candy, cookie, biscuit, hot cake, chocolate, and rice cake; kneaded marine products represented by (boiled) fish cake; live-stock products represented by ham, sausage, hamburger and steak; daily dishes such as cream croquette, paste for Chinese foods, gratin, and dumpling; foods of delicate flavor, such as salted fish guts and a vegetable pickled in sake lee; liquid diets such as tube feeding liquid food; supplements; and pet foods. Whereas traditional processes for preparing food and feed products will require an emulsification or homogenization step, the process of the current invention for preparing the food and feed products does not need such a homogenization or emulsification step. A simple blending of the oleosome composition with the other ingredients is sufficient.

Examples of such pharmaceutical products include products comprising therapeutic agents, diagnostic agents, and delivery agents. As a therapeutic or diagnostic agent, the product will additionally contain an active ingredient. The active ingredient can be anything that one wishes to deliver to a host. The active ingredient may be a protein or peptide that has therapeutic or diagnostic value. Such peptides include antigens (for vaccine formulations), antibodies, cytokines, blood-clotting factors, and growth hormones. An example of a pharmaceutical product is a parenteral emulsion containing the oleosome composition and a drug.

Examples of such personal care products include soaps, cosmetics, skin creams, facial creams, toothpaste, lipstick, perfumes, make-up, foundation, blusher, mascara, eyeshadow, sunscreen lotions, hair conditioner, and hair coloring.

Examples of such industrial products include paints, coatings, lubricants, films, gels, drilling fluids, paper sizing, latex, building, and road construction material, inks, dyes, waxes, polishes, and agrochemical formulations.

Furthermore, the current invention relates to a process for preparing the product according to the invention comprising the oleosome composition; the process comprising the step of blending the oleosome composition with the at least one other ingredient other than oleosomes.

The process preferably does not comprise a further step of emulsification of the oleosome composition with the at least one other nutritional composition.

### Process for Preparing the Oleosome Composition

The present invention relates to a process for preparing the oleosomes composition, and the process comprises the steps of:
a) blending one or more sources of triglycerides having saturated C16 fatty acids that are positioned at the sn2 position, and optionally other lipids with isolated vegetable oleosomes in a ratio of the source of triglycerides having saturated C16 fatty acids that are positioned at the sn2 position and optionally other lipids to oleosomes of from 1:99 to 95:5; and
b) subjecting the blend obtained from step a) to a high-shear force and obtaining an oleosome composition comprising loaded isolated vegetable oleosomes being isolated vegetable oleosomes loaded with one or more sources of triglycerides having saturated C16 fatty acids that are positioned at the sn2 position, which are present in the inside of the loaded oleosomes and wherein at least 80 wt. % of the total weight of lipids in the composition is present in the oleosomes.

In one aspect, the present invention relates to a process for preparing the oleosomes composition, and the process comprises the steps of:
a) blending one or more sources of triglycerides having saturated C16 fatty acids that are positioned at the sn2 position and other lipids with isolated vegetable oleosomes in a ratio of the source of triglycerides having saturated C16 fatty acids that are positioned at the sn2 position and other lipids to oleosomes of from 1:99 to 95:5; and
b) subjecting the blend obtained from step a) to a high-shear force and obtaining an oleosome composition comprising loaded isolated vegetable oleosomes being isolated oleosomes loaded with one or more sources of triglycerides having saturated C16 fatty acids that are positioned at the sn2 position, which are present in the inside of the loaded oleosomes and wherein at least 80 wt. % of the total weight of lipids in the composition is present in the oleosomes.

In another aspect, the present invention relates to a process for preparing the oleosomes composition, and the process comprises the steps of:
a) blending one or more sources of triglycerides having saturated C16 fatty acids that are positioned at the sn2 position with isolated vegetable oleosomes in a ratio of the source of triglycerides having saturated C16 fatty acids that are positioned at the sn2 position to oleosomes of from 1:99 to 95:5; and
b) subjecting the blend obtained from step a) to a high-shear force and obtaining an oleosome composition comprising loaded isolated vegetable oleosomes being isolated vegetable oleosomes loaded with one or more sources of triglycerides having saturated C16 fatty acids that are positioned at the sn2 position, which are present in the inside of said loaded oleosomes and wherein at least 80 wt. % of the total weight of lipids in the composition is present in the oleosomes.

The isolated vegetable oleosomes comprise lipids that are by nature present in vegetable seeds and the oleosomes are isolated from the seeds. These isolated vegetable oleosomes are loaded—according to the process of the invention—with one or more lipids to obtain an oleosome composition comprising loaded isolated vegetable oleosomes. The loading (=blending and subjecting to high-shear force) with specific lipids allows obtaining oleosomes comprising lipids with a specific fatty acid profile. The additional lipids added are (mostly) present inside the loaded isolated vegetable oleosomes. In other words, at least 80 wt. % of the total weight of lipids in the oleosome composition that is obtained from the process, is present in the loaded isolated vegetable oleosomes. Preferably, at least 90 wt. % or at least 95 wt. % or at least 98 wt. % of the total weight of lipids in the oleosome composition is present in the loaded isolated vegetable oleosomes. In fact, from 96 wt. % to 99.8 wt. %, from 97 wt. % to 99.0 wt. % of the total weight of lipids in the composition is present in the oleosomes.

More details regarding the several steps of the process are disclosed below.

### Step a)

The process starts with isolated vegetable oleosomes or enlarged isolated vegetable oleosomes and blending these with one or more sources of triglycerides having saturated C16 fatty acids that are positioned at the sn2 position (being one or more lipids). The ratio between the source of triglycerides having saturated C16 fatty acids that are positioned at the sn2 position and the oleosomes . . . preferably the ratio between the source of triglycerides having saturated C16 fatty acids that are positioned at the sn2 position to the lipids of the oleosomes (=the lipids already present in the oleosomes) is from 1:99 to 95:5, preferably from 5:95 to 80:20, more preferably from 10:90 to 60:40. Optionally, other lipids may also be blended with the isolated vegetable oleosomes for the purpose of being loaded into the isolated oleosomes. When also other lipids are blended with the isolated vegetable oleosomes, the ratio between the one or more sources of triglycerides having saturated C16 fatty acids that are positioned at the sn2 position and other lipids to oleosomes is from 1:99 to 95:5, preferably from 5:95 to 80:20, more preferably from 10:90 to 60:40.

Methods for obtaining isolated oleosomes are well known in the art. Typically, vegetable seeds are obtained using agricultural cultivation practices well known to a person skilled in the art. The seeds are harvested and, if desired, materials such as stones or seed hulls (de-hulling) may be removed from the seeds by, for example, sieving or rinsing. Subsequently the seeds are processed by mechanical pressing, grinding or crushing. A liquid phase, e.g. water, may also be added prior to grinding of the seeds, which is known as wet milling. Following grinding, a slurry is obtained and filtrated. The filtrate may be subsequently separated into two liquid phases, a watery phase and an oily oleosome containing phase, i.e. the isolated vegetable oleosomes, by means of any suitable separation technique such as, but not limited to centrifugal acceleration. Alternatively, the slurry obtained after grinding may be submitted to a liquid-solid separation (two-phase separation) or a liquid-solid-liquid separation (three-phase separation) using a centrifugal decanter. Both separation techniques follow the same operating principle.

The isolated vegetable oleosomes or enlarged isolated vegetable oleosomes in step a) may have a dry matter content in a range of from 30 to 80 wt. % on the total weight of the oleosomes, the remainder up to 100 wt. % being an aqueous solution such as but not limited to water. The isolated oleosomes that are subjected to step a) may have a pH in a range of from 3.5 to 10.0, preferably from 4.5 to 8.5, more preferably from 5.5 to 7.5.

The isolated sunflower oleosomes used in step a) of the process of the present invention may be washed or non-washed isolated sunflower oleosomes.

Briefly stated, enlarged isolated vegetable oleosomes may be obtained by carrying out a process of applying high-shear centrifugation force to the isolated oleosomes and/or a process of applying high-shear mixing to the isolated oleosomes, such as described in WO2021126408A1 by the present applicant.

Isolated vegetable oleosomes or enlarged isolated vegetable oleosomes that are subjected to step a) of the process according to the invention, may be in liquid form or in dehydrated form. When these vegetable oleosomes are in dehydrated form, they are prior to step a) suspended in an aqueous solution, such as but not limited to water, in order to have a dry matter content in a range of from 30 to 80 wt. % on the total weight of the oleosomes.

### Step b)

The blend obtained from step a) is subjected to a high-shear force to obtain an oleosome composition comprising loaded isolated vegetable oleosomes being isolated vegetable oleosomes loaded with one or more sources of triglycerides having saturated C16 fatty acids that are positioned at the sn2 position, which are present in the inside of the loaded oleosomes and wherein at least 80 wt. % of the total weight of lipids in the composition is present in the oleosomes.

The high-shear force to which the blend obtained in step a) is subjected, may be a high-shear mixing, a high-pressure homogenization, an (ultra) sonication, or a hydrodynamic cavitation mixing. The high shear mixing in step b) may be applied for a period of time in a range of from 0.5 to 10 minutes, preferably 1 to 8 minutes, more preferably 2 to 6 minutes. The high-pressure homogenization may be carried out with a pressure up to 300 bar, resulting in a smooth and stable composition. In case the high-shear force in step b) is obtained using high-shear mixing, the high-shear mixing may be applied using a rotor-stator high-shear mixer at a tip velocity in a range of from 1.6 to 12.8 m/s, preferably 1.9 to 11.2 m/s, more preferably 2.6 to 9.6 m/s.

It is surprisingly found that high shear is applicable to obtain the loaded isolated oleosomes in the oleosome composition of the present invention. The oleosomes are still intact and the loaded material is present in the inside of the oleosomes.

In one aspect of the invention, the process for preparing the oleosomes composition, comprises the steps of:
a) blending one or more sources of triglycerides having saturated C16 fatty acids that are positioned at the sn2 position and optionally other lipids with isolated vegetable oleosomes in a ratio of the source of triglycerides and optionally other lipids to oleosomes of from 1:99 to 95:5; and
b) subjecting the blend obtained from step a) to a high-shear mixing to obtain an oleosome composition comprising isolated vegetable oleosomes loaded with one or more sources of triglycerides having saturated C16 fatty acids that are positioned at the sn2 position, wherein at least 80 wt. % of the total weight of lipids in the composition is present in the oleosomes,
   wherein the high-shear mixing is applied
for a period of time in a range of from 0.5 to 10 minutes, preferably 1 to 8 minutes, more preferably 2 to 6 minutes, and
using a rotor-stator high-shear mixer at a tip velocity in a range of from 1.6 to 12.8 m/s, preferably 1.9 to 11.2 m/s, more preferably 2.6 to 9.6 m/s

Amongst others it has been found that the process for preparing the oleosomes of the present invention is more convenient, and/or more simple than the process for preparing the synthetic emulsion. The process allows to get a homogeneous product, without or with a very limited amount free lipids present, whereas the amount of free lipids is significant for the synthetic emulsions.

### Additional Steps

### Washing Step

A washing step may be included between step a) and b).

The product obtained in step a may be washed by re-suspending it in a floatation solution of lower density (e.g. water, aqueous buffer with neutral to alkaline pH up to 9.5) and by subsequently separating it again from the aqueous phases by means of any suitable separation technique such as, but not limited to centrifugation. The washing procedure may be repeated several times, from one up to three times.

### Heat-Treatment Step

The isolated vegetable oleosomes or enlarged vegetable oleosomes may be heat-treated prior to step a) or after step b) of the process according to the invention. Furthermore, the oleosome composition obtained in step b) may be subjected to a heat treatment step.

The heat treatment may be a pasteurization treatment or an ultra-high-temperature (UHT) treatment. Pasteurization treatment involves heating the oleosome composition at 65° C. to 70° C. for 30 minutes in batch, or 80° C. to 86° C. for 15 to 30 seconds in a continuous-flow process (High-temperature short time Pasteurization (HTST Pasteurization)). UHT treatment involves heating of the oleosome composition at a temperature of 135° C. to 150° C. in a continuous-flow process and holding at that temperature for one or more seconds, up to 5 seconds, before cooling rapidly to room temperature. The heat treatment step of the oleosome composition is applied to further avoid microbial contamination of the oleosomes.

### Dehydration Step

The oleosome composition obtained in step b) may be subjected to a dehydration step. Dehydration steps well known to the person skilled in the art are amongst others spray drying, fluid bed drying, freeze-drying, and vacuum drying. The thus obtained oleosomes are in a more concentrated liquid form or a powder form. In one aspect of the invention, the dehydration step is a spray-drying step.

### Enlargement Step

The isolated vegetable oleosomes may be subjected to a step of enlarging the oleosomes prior to step a) of the process according to the invention. Furthermore, the product obtained prior to step b) may be subjected to a step of enlarging the oleosomes to form enlarged oleosomes. This may be carried out by a process of applying high-shear centrifugation force to the isolated oleosomes and/or a process of applying high-shear mixing to the isolated oleosomes, such as described in WO2021126408A1 by the present applicant.

Alternatively, the step of enlarging and loading the isolated vegetable oleosomes can be done simultaneously. For simultaneously loading and enlarging isolated vegetable oleosomes, the high-shear force in step b) may be obtained by using high-shear mixing that is applied for a period of time in a range of 3.0 to 10.0 minutes, preferably 3.5 to 8.0 minutes, more preferably 4.0 to 6.0 minutes.

Preferably, for simultaneously loading and enlarging isolated vegetable oleosomes, the high-shear force in step b) is applied to isolated vegetable oleosomes that have been washed prior to step a).

### Use of an Oleosome Composition

Finally, the invention relates to the use of the oleosome composition as a carrier for triglycerides having saturated C16 fatty acids that are positioned at the sn2 position.

More specifically, the oleosome composition can be used for the prevention of oxidation of triglycerides having saturated C16 fatty acids that are positioned at the sn2 position that is loaded into isolated vegetable oleosomes and/or improving the stability of the triglycerides having saturated C16 fatty acids that are positioned at the sn2 position in the gastric phase of the human digestive tract.

The current invention relates to the use of the oleosome composition as a carrier providing oxidative stability while allowing the tailoring of the fatty acid profile according to the desired need.

### Effects of the Invention

The present invention provides a composition having a specific fatty acid profile using an oleosome composition comprising loaded isolated vegetable oleosomes being isolated vegetable oleosomes loaded with one or more sources of triglycerides having saturated C16 fatty acids that are positioned at the sn2 position which are present in the inside of the loaded oleosomes. The oleosome composition of the present invention provides a matrix that offers improved protection of triglycerides having saturated C16 fatty acids that are positioned at the sn2 position against oxidation and/or improved stability in the gastric phase of the human digestive tract. Additionally, nutritional compositions wherein at least 90%, or even substantially all of the lipids are present in the oleosome composition, will be stable without the need of any emulsifier. The simpler process resulted further in a more stable product having a longer shelf life and/or having less free lipids in the oleosome composition than in the synthetic emulsions.

### CLAUSES

1. An oleosome composition comprising loaded isolated vegetable oleosomes being isolated vegetable oleosomes loaded with one or more sources of triglycerides having saturated C16 fatty acids that are positioned at the sn2 position, which are present in the inside of said loaded isolated vegetable oleosomes.

2. An oleosome composition comprising loaded isolated vegetable oleosomes being isolated vegetable oleosomes loaded with one or more sources of triglycerides having saturated C16 fatty acids that are positioned at the sn2 position, which are present in the inside of said loaded isolated vegetable oleosomes and wherein at least 80 wt. % of the total weight of lipids in the composition is present in the oleosomes.

3. The oleosome composition according to clause 1 or 2, wherein the oleosomes are further loaded with one or more sources of long-chain polyunsaturated fatty acids (LC-PUFA) and/or with one or more sources of medium-chain fatty acids (MCFA).

4. The oleosome composition according to any one of the preceding clauses, wherein the oleosomes are further loaded with one or more sources of long-chain polyunsaturated fatty acids (LC-PUFA), ALA, and/or with one or more sources of medium-chain fatty acids (MCFA).

5. The oleosome composition according to any one of the preceding clauses, wherein poly-unsaturated fatty acids (LC-PUFA) have a chain (“backbone”) comprising 20 or more carbon atoms and comprise more than one double bond in their backbone.

6. The oleosome composition according to any one of the preceding clauses wherein LC-PUFA fatty acids are selected from the group consisting of arachidonic acid, docosahexaenoic acid, eicosapentaenoic acid, docosapentaenoic acid, or mixtures of two or more thereof.

7. The oleosome composition according to any one of the preceding clauses wherein medium-chain fatty acids are selected from the group consisting of saturated fatty acids having a carbon chain length of 6, 8, 10, or 12 carbon atoms or mixtures of wo or more thereof.

8. The oleosome composition according to any one of the preceding clauses wherein medium-chain fatty acids MCFA are sourced from lipid sources selected from coconut oil, palm kernel oil, dairy lipids, fractions or mixtures thereof.

9. The oleosome composition according to any one of the preceding clauses, wherein said isolated vegetable oleosomes are selected from the group consisting of isolated rapeseed oleosomes, isolated soybean oleosomes, isolated sunflower oleosomes, isolated linseed oleosomes, and a combination of two or more thereof.

10. The oleosome composition according to any one of the preceding clauses comprising loaded isolated soy bean oleosomes being isolated soy bean oleosomes loaded with one or more sources of triglycerides having saturated C16 fatty acids that are positioned at the sn2 position, which are present in the inside of said loaded isolated soybean oleosomes, and wherein at least 80 wt. % of the total weight of lipids of the composition is present in the oleosomes.

11. The oleosome composition according to any one of the preceding clauses comprising loaded isolated rapeseed oleosomes being isolated rapeseed oleosomes loaded with one or more sources of triglycerides having saturated C16 fatty acids that are positioned at the sn2 position which are present in the inside of said loaded isolated rapeseed oleosomes, and wherein at least 80 wt. % of the total weight of lipids of the composition is present in the oleosomes.

12. The oleosome composition according to any one of the preceding clauses comprising loaded isolated linseed oleosomes being isolated linseed oleosomes loaded with one or more sources of triglycerides having saturated C16 fatty acids that are positioned at the sn2 position which are present in the inside of said loaded isolated linseed oleosomes, and wherein at least 80 wt. % of the total weight of lipids of the composition is present in the oleosomes.

13. The oleosome composition according to any one of the preceding clauses comprising loaded isolated sunflower oleosomes being isolated sunflower oleosomes loaded with one or more sources of triglycerides having saturated C16 fatty acids that are positioned at the sn2 position which are present in the inside of said loaded isolated sunflower oleosomes, and wherein at least 80 wt. % of the total weight of lipids of the composition is present in the oleosomes.

14. The oleosome composition according to any one of the preceding clauses, comprising the one or more sources of triglycerides having saturated C16 fatty acids that are positioned at the sn2 position in an amount of from 0.5 to 90.0 wt. %, preferably from 1.0 to 80.0 wt. %, more preferably from 1.5 to 70 wt. % based on the total dry weight of the oleosome composition.

15. The oleosome composition according to any one of the preceding clauses, comprising the one or more sources of triglycerides having saturated C16 fatty acids that are positioned at the sn2 position in an amount of from 0.5 to 50.0 wt. %, preferably from 1.0 to 40.0 wt. %, more preferably from 1.5 to 30 wt. % based on the total dry weight of the oleosome composition.

16. The oleosome composition according to any one of the preceding clauses, wherein the one or more sources of triglycerides having saturated C16 fatty acids that are positioned at the sn2 position has a fatty acid profile comprising from 30 to 50% by weight of saturated fatty acids having 16 carbon atoms and from 30 to 70% by weight of unsaturated fatty acids having 18 carbon atoms based on the total weight of the fatty acid profile of the one or more source, wherein 50 to 80%, such as from 52 to 75% by weight of the saturated fatty acids having 16 carbon atoms are present at the 2-position of a triglyceride.

17. The oleosome composition according to any one of the preceding clauses, comprising an amount of saturated C16 fatty acids that is positioned at the sn2 position of a triglyceride is in a range of from 0.075 to 36.0 wt. %, such as from 0.1 to 36.0 wt. %, preferably from 0.15 to 32 wt. %, such as 0.2 to 32.0 wt. %, more preferably 0.5 to 28.0 wt. % or from 0.234 to 26.25 wt. %, based on the total weight of the fatty acid profile of the oleosome composition.

18. The oleosome composition according to any of the preceding clauses, further comprising one or more sources of one or more lipophilic dietary bioactive substances, selected from the group consisting of oil-soluble vitamins, phytosterols, curcuminoids, carotenoids, and flavonoids, and combinations of two or more thereof.

19. The oleosome composition according to any one of the preceding claims, wherein the one or more lipophilic dietary bioactive substance is selected from the group consisting of vitamin D, vitamin E, vitamin A, vitamin K, and combinations of two or more thereof.

20. The oleosome composition according to any one of the preceding clauses, wherein the lipophilic dietary bioactive substance is vitamin D, preferably wherein vitamin D is present as a lipophilic dietary bioactive substance; preferably wherein the content of vitamin D is in a range of from 0.1 to 1000 microgram per gram, based on the total weight of lipids of the oleosome composition.

21. The oleosome composition according to any one of the preceding clauses, wherein the lipophilic dietary bioactive substance is vitamin A.

22. The oleosome composition according to any one of the preceding clauses, wherein the lipophilic dietary bioactive substance is vitamin E.

23. The oleosome composition according to any one of the preceding clauses, wherein the lipophilic dietary bioactive substance is vitamin K.

24. The oleosome composition according to any one of the preceding clauses, wherein the oleosomes have an average globule diameter in a range of from 2.5 to 15 microns, preferably from 3.0 to 12 microns, more preferably from 4.0 to 10 microns.

25. An oleosome composition comprising loaded isolated vegetable oleosomes prepared by
a) blending one or more sources of one or more triglycerides having saturated C16 fatty acids that are positioned at the sn2 position and
b) subjecting the blend obtained from step a) to a high-shear force and obtaining an oleosome composition comprising loaded isolated vegetable oleosomes loaded with one or more sources of one or more lipophilic dietary bioactive substances, which are present in the inside of said loaded isolated vegetable oleosomes, and wherein at least 80 wt. % of the total weight of lipids in the composition is present in the oleosomes.

26. An oleosome composition comprising loaded isolated vegetable oleosomes prepared by
a) blending one or more sources of one or more triglycerides having saturated C16 fatty acids that are positioned at the sn2 position and
b) subjecting the blend obtained from step a) to a high-shear force and obtaining an oleosome composition comprising loaded isolated vegetable oleosomes loaded with one or more sources of one or more lipophilic dietary bioactive substances, which are present in the inside of said loaded isolated vegetable oleosomes.

27. An oleosome composition comprising loaded isolated vegetable oleosomes prepared by
a) blending one or more sources of lipophilic dietary bioactive substances, ALA, LC-PUFA, MCFA's 1,3-dioleoyl-2-palmitoyl glyceride (OPO), 1,3-dilinoleoyl-2-palmitoyl glyceride (LPL), 1-oleoyl-2-palmitoyl-3-linoleoyl glyceride (OPL) or mixture of two or more thereof with isolated vegetable oleosomes, and
b) subjecting the blend obtained from step a) to a high-shear force and obtaining an oleosome composition comprising loaded isolated vegetable oleosomes loaded with one or more sources of lipophilic dietary bioactive substances. ALA, LC-PUFA, 1,3-dioleoyl-2-palmitoyl glyceride (OPO), 1,3-dilinoleoyl-2-palmitoyl glyceride (LPL), 1-oleoyl-2-palmitoyl-3-linoleoyl glyceride (OPL) or mixture of two or more thereof and optionally other lipids, which are present in the inside of said loaded isolated vegetable oleosomes, and wherein at least 80 wt. % of the total weight of lipids in the composition is present in the oleosomes, and wherein the lipophilic dietary bioactive substances are selected from the group consisting of oil-soluble vitamins, phytosterols, curcuminoids, carotenoids, and flavonoids, and combinations of two or more thereof.

28. An oleosome composition comprising loaded isolated vegetable oleosomes prepared by
a) blending one or more sources of lipophilic dietary bioactive substances, ALA, LC-PUFA, MCFA's 1,3-dioleoyl-2-palmitoyl glyceride (OPO), 1,3-dilinoleoyl-2-palmitoyl glyceride (LPL), 1-oleoyl-2-palmitoyl-3-linoleoyl glyceride (OPL) or mixture of two or more thereof with isolated vegetable oleosomes, and
b) subjecting the blend obtained from step a) to a high-shear force and obtaining an oleosome composition comprising loaded isolated vegetable oleosomes loaded with one or more sources of lipophilic dietary bioactive substances, ALA, LC-PUFA, 1,3-dioleoyl-2-palmitoyl glyceride (OPO), 1,3-dilinoleoyl-2-palmitoyl glyceride (LPL), 1-oleoyl-2-palmitoyl-3-linoleoyl glyceride (OPL) or mixture of two or more thereof and optionally other lipids, which are present in the inside of said loaded isolated vegetable oleosomes, and wherein the lipophilic dietary bioactive substances are selected from the group consisting of oil-soluble vitamins, phytosterols, curcuminoids, carotenoids, and flavonoids, and combinations of two or more thereof.

29. An oleosome composition comprising loaded isolated vegetable oleosomes prepared by
a) blending one or more sources of lipophilic dietary bioactive substances. ALA, LC-PUFA, MCFA's 1,3-dioleoyl-2-palmitoyl glyceride (OPO), 1,3-dilinoleoyl-2-palmitoyl glyceride (LPL), 1-oleoyl-2-palmitoyl-3-linoleoyl glyceride (OPL) or mixture of two or more thereof with isolated vegetable oleosomes, and
b) subjecting the blend obtained from step a) to a high-shear force and obtaining an oleosome composition comprising loaded isolated vegetable oleosomes loaded with one or more sources of lipophilic dietary bioactive substances, ALA, LC-PUFA, 1,3-dioleoyl-2-palmitoyl glyceride (OPO), 1,3-dilinoleoyl-2-palmitoyl glyceride (LPL), 1-oleoyl-2-palmitoyl-3-linoleoyl glyceride (OPL) or mixture of two or more thereof and optionally other lipids, which are present in the inside of said loaded isolated vegetable oleosomes, and wherein at least 80 wt. % of the total weight of lipids in the composition is present in the oleosomes, and wherein the lipophilic dietary bioactive substances are selected from the group consisting of vitamin D, vitamin E, vitamin A, vitamin K, and combinations of two or more thereof.

30. An oleosome composition comprising loaded isolated vegetable oleosomes prepared by
a) blending one or more sources of lipophilic dietary bioactive substances, ALA, LC-PUFA, MCFA's 1,3-dioleoyl-2-palmitoyl glyceride (OPO), 1,3-dilinoleoyl-2-palmitoyl glyceride (LPL), 1-oleoyl-2-palmitoyl-3-linoleoyl glyceride (OPL) or mixture of two or more thereof with isolated vegetable oleosomes, and
b) subjecting the blend obtained from step a) to a high-shear force and obtaining an oleosome composition comprising loaded isolated vegetable oleosomes loaded with one or more sources of lipophilic dietary bioactive substances, ALA, LC-PUFA, 1,3-dioleoyl-2-palmitoyl glyceride (OPO), 1,3-dilinoleoyl-2-palmitoyl glyceride (LPL), 1-oleoyl-2-palmitoyl-3-linoleoyl glyceride (OPL) or mixture of two or more thereof and optionally other lipids, which are present in the inside of said loaded isolated vegetable oleosomes, and wherein the lipophilic dietary bioactive substances are selected from the group consisting of vitamin D, vitamin E, vitamin A, vitamin K, and combinations of two or more thereof.

31. The oleosome composition according to any of the preceding clauses, wherein the oleosome composition is containing from 0.2 to 10 wt. %, preferably from 1.2 to 8 wt. %, more preferably from 1.5 to 6 wt % of proteins based upon the dry weight of the composition.

32. A product comprising the oleosome composition according to any one of the preceding clauses, wherein said product is selected from the group consisting of food products, feed products, pharmaceutical products, personal care products, nutritional compositions and industrial products.

33. The product according to clause 32, being an infant formula, preferably growing-up milk.

34. The product according to clause 33, wherein the infant formula has a total content of lipids in a range of from 2.5 to 4.5 wt. %, from 2.8 to 4.0 wt. %, or from 3.0 to 3.4 wt. % on total weight of the infant formula, wherein at least 90%, at least 95%, or even substantially all of the lipids of the infant formula is present in the oleosome composition, and wherein the oleosome composition

is comprising isolated vegetable oleosomes loaded with one or more source of triglycerides having saturated C16 fatty acids that are positioned at the sn2 position; and is comprising an amount of triglycerides having saturated C16 fatty acids that are positioned at the sn2 position of a triglyceride in a range of from 5.0 to 25.0 wt. %, from 8.0 to 20.0 wt. %, or from 10.0 to 18.0 wt. % based on the total weight of the fatty acid profile of the oleosome composition, and wherein at least 80 wt. % of the total weight of lipids in the oleosome composition is present in the oleosomes.

35. A process for loading isolated vegetable oleosomes, wherein the process comprises the steps of:
a) blending one or more sources of triglycerides having saturated C16 fatty acids that are positioned at the sn2 position, and optionally other lipids with isolated vegetable oleosomes in a ratio of the source of triglycerides having saturated C16 fatty acids that are positioned at the sn2 position and optionally other lipids to oleosomes of from 1:99 to 95:5; and
b) subjecting the blend obtained from step a) to a high-shear force and obtaining an oleosome composition comprising loaded isolated vegetable oleosomes being isolated vegetable oleosomes loaded with one or more sources of triglycerides having saturated C16 fatty acids that are positioned at the sn2 position, present in the inside of the loaded oleosomes and wherein at least 80 wt. % of the total weight of lipids in the composition is present in the oleosomes.

36. The process according to clause 35, wherein the isolated oleosomes in step a) have a dry matter content in a range of from 30 to 80 weight % on the total weight of the oleosomes.

37. Use of the oleosome composition according to any one clauses 1 to 31 or prepared according to the process according to clause 35 to 36 as a carrier for triglycerides having saturated C16 fatty acids that are positioned at the sn2 position.

38. A process for preparing a product comprising the oleosome composition according to any one of the clauses 1 to 31, wherein the product is comprising at least one further ingredient different from oleosomes and the product is selected from the group consisting of food products, feed products, pharmaceutical products, personal care products, nutritional compositions, and industrial products, and the process comprises adding the oleosome composition according to any one of clauses 1 to 31, to at least one further ingredient different from oleosomes wherein the oleosome composition is added in an amount in a range of from 0.1 to 70.0 wt. % on dry matter of the product.

39. A process for preparing a product according to any one of the clauses 32 to 34, wherein the product is selected from the group consisting of food products, feed products, pharmaceutical products, personal care products, nutritional compositions, and industrial products and the process comprises adding the oleosome composition according to any one of clauses 1 to 31, to at least one further ingredient different from oleosomes wherein the oleosome composition is added in an amount in a range of from 0.1 to 70.0 wt. % on dry matter of the product.

### EXAMPLES

The present invention is further elucidated based on the Examples below which are illustrative only and not considered limiting to the present invention.

### Analysis Methods

### Protein Measurement

The protein content of the oleosomes was determined by the amount of nitrogen in the sample. This amount of nitrogen was analyzed using a combustion method. Combustion of the sample was performed at 1100° C. The amount of nitrogen was determined using a conductivity detector (LECO TruMAc). The protein content was calculated by multiplying the amount of nitrogen analyzed by 6.25.

### Measurement of Fat Content

The amount of fat (lipids) in the isolated oleosomes was determined using the Soxhlet extraction method. It is expressed on total dry weight of the isolated oleosomes.

### Measurement of Dry Weight

The percentage dry substance (% DS) of the isolated/loaded oleosomes was determined gravimetrically using an MA150 infrared balance (Sartorius). About 2 g of material (i.e; the wet weight (WW)) is applied on an aluminum dish with glass fiber pad. The moisture is evaporated at 105° C. until a stable weight is reached (i.e; the dry weight (DW)). The percentage dry substance is calculated according to the following formula:

$$\% DS = \frac{DW}{WW} \times 100$$

### Measurement of Free Oil in Oleosome Composition

A pre-weighted sample of loaded isolated vegetable composition was mixed with heptane in a ratio of 1:5 sample; heptane and agitated for 15 minutes for extraction of the free oil into the heptane phase. After extraction the solution is centrifuged for 5 minutes followed by filtration of the top layer through a 0.45 μm filter. The free oil was quantified by GPC.

### Measurement of Oil Present in Oleosomes in the Oleosomes Composition

The amount of oil present in the loaded isolated oleosomes of the oleosome composition was calculated by subtracting the amount of free oil from the total oil content of the oleosome composition.

### Size Measurement of Isolated Vegetable Oleosomes and Loaded Isolated Vegetable Oleosomes

To be able to measure the globule diameter of the oleosomes, a Mastersizer 3000 from Malvern equipped with a Hydro module was used during the measurements. A refractive index of 1.47, a dispersant refractive index of 1.33 and a particle absorption index of 0.01 is used to measure the oleosomes size. The concentration of the oleosomes in the buffer is such that an obscuration in the range from 8.0 to 8.5% in the Mastersizer equipment will be obtained. Obscuration within the Mastersizer is the amount of light blocked or scattered, by the particles. Therefore, the oleosomes are diluted in a buffer solution containing 10 mM sodium phosphate, pH 7.4, and 1.0 wt. % sodium dodecyl sulfate (SDS). For example, about 0.2 wt. % of oleosomes is diluted in the buffer solution and the dilution is further adjusted to obtain the obscuration. Once this optimal obscuration is obtained, the globule diameter is measured, and the average globule diameter (D50-value) can be calculated.

### Determination of Oxidation Stability

The oxidation stability was determined by measuring the Oxidation Induction Period (OIP) was using an ML Oxipres device (Mikrolab). Sample were subjected to a high oxidative-stress environment in order to evaluate, in a short period of time, the resistance to oxidation. The oxygen uptake of the reactive components present in the samples was monitored as a pressure drop in function of time. An amount of sample (11-12 g) (i.e. isolated oleosomes or an oleosome composition) equivalent to 4 g of fat was brought into a glass vessel which was placed in a pre-heated (70° C.) pressure vessel and filled with oxygen to 5 bar. The OIP was determined as the intersection between the two tangents before and after the inflection point on the pressure graph.

### Obtaining Isolated Sunflower Oleosomes

In order to isolate oleosomes from sunflower, 100 g of dehulled seeds from (high-oleic) sunflower were soaked during 2 hours in deionized water in a ratio of seeds to water of 1:3 at a temperature of 20° C. The soaking water was discarded and the soaked seeds were washed with deionized water in a ratio of seeds to water of 1:2 at a temperature of 20° C. The washing water was discarded and the washed seeds were ground with deionized water in a ratio of seeds to water of 1:10 at a temperature of 20° C. using a Thermomix® TM5 (Vorwerk) at a speed of 10700 rpm for 90 seconds. The obtained slurry was filtered over a Nylon filter with a pore diameter of 80 microns to obtain a filtrate, the retentate was discarded. The pH of the filtrate was adjusted to 7.5 with a sodium hydroxide solution and subsequently centrifuged for a first time using a Thermo Scientific Sorvall Legend at 4950 g for 30 minutes, to yield a solid pellet (cell debris and insoluble proteins), a hydrophilic liquid supernatant phase (watery solution of proteins, carbohydrates and soluble fiber), and a hydrophobic creamy top layer with the desired oleosomes. The solid pellet and supernatant were discarded and the creamy top layer was diluted to 15% dry substance with deionized water, brought to pH 9.5 with a sodium hydroxide solution and centrifuged for a second time at 4950 g for 30 minutes (Thermo Scientific Sorvall Legend), to yield a liquid supernatant phase and a creamy top layer with the desired isolated (high-oleic) sunflower oleosomes, which were collected. The amount of proteins in the isolated (high-oleic) sunflower oleosomes was 1.9 wt. % on basis of the dry weight of oleosomes, using the below described method. The amount of fat in the isolated (high-oleic) sunflower oleosomes was 35 wt. % on basis of the total weight of oleosomes, using the previous described method. The amount of fat (lipids) in the isolated (high-oleic) sunflower oleosomes can be expressed on dry weight of oleosomes and was 97.2 wt. %. The difference up to 100% dry weight represents traces of other components (not shown here). The composition is provided in Table 3.

### Loading of the Isolated Oleosomes

Step a) Blending One or More Sources of Triglycerides Having Saturated C16 Fatty Acids that Are Positioned at the Sn2 Position with Isolated Oleosomes

In order to obtain an oleosome composition comprising loaded isolated vegetable oleosomes having a targeted fatty acid profile and targeted triglyceride composition, isolated vegetable oleosomes were blended in step a) with additional oils as a source of triglycerides having saturated C16 fatty acids that are positioned at the sn2 position and optionally specific lipids including specific fatty acids. The targeted fatty acid profile and targeted triglyceride composition was that of the infant formula wherein the oleosome composition was used as a source of a part of the fats (lipids).

The isolated oleosomes were blended with a blend of liquid oils, being OPO-oil (sn-2 palmitate oil) as a source of triglycerides having saturated C16 fatty acids that are positioned at the sn2 position, rapeseed oil as a source of linoleic acid, and ALA, Schizochytrium spec. oil as a source of DHA (LC-PUFA), and Mortierella alpina oil as a source of ARA (LC-PUFA). The ratio of the amount of isolated oleosomes to the amount of source of triglycerides having saturated C16 fatty acids that are positioned at the sn2 position and optionally other lipids is between 39 to 61 and 15 to 85. The liquid oils (according to Table 1) were added to the isolated oleosomes by gentle mixing using a spatula, until the oil is finely dispersed.

TABLE 1
Recipes for blending of isolated oleosomes with one
or more sources of triglycerides having saturated C16
fatty acids that are positioned at the sn2 position

| Isolated | Lipid | Example | Example |
|---|---|---|---|
| oleosomes | phase | 1 | 2 |
| High oleic | High oleic sunflower oil | 31 wt. % | 15 wt. % |
| sunflower | contributed by addition | | |
| oleosomes* | of oleosomes | | |
| | Rapeseed oil | 20 wt. % | none |
| | (source of ALA) | | |
| | OPO oil (sn-2 | 45 wt. % | 85 wt. % |
| | palmitate oil) | | |
| | (source of triglycerides | | |
| | saturated C16 fatty | | |
| | acids @ sn2) | | |
| | Schizochytrium spec. oil | 2 wt. % | none |
| | (source of LC-PUFA - DHA) | | |
| | Mortierella Alpina oil | 2 wt. % | none |
| | (source of LC-PUFA -ARA) | | |
| | Total amount | 100 wt. % | 100 |
| | Ratio of oil present in isolated | 31:69 | 15:85 |
| | oleosomes to source of | | |
| | triglycerides having saturated | | |
| | C16 fatty acids that are | | |
| | positioned at the sn2 position | | |
| | and optionally other lipids | | |

The fatty acid composition of the different oils used for the loading (as well as high-oleic sunflower oil) are shown in Table 2 for the fatty acids that are present in 1% or more; the numbers hence do not add up to 100%. Where totals are given, the fatty acids that are present in less than 1% are also included even if not specifically mentioned in the table below; the sum of the individual numbers therefore do not always add up to the total number.

The fatty acid composition of the isolated and the loaded isolated oleosomes, according to Example 1 is specified in Table 3.

TABLE 2
Fatty acid profile of oils used for blending with the isolated oleosomes

| | Fatty | | | Mortierella | |
|---|---|---|---|---|---|
| | acid | Rapeseed | OPO- | Alpina | Schizochytrium |
| Fatty acid | name | oil* | oil*** | oil** | Oil* |
| C 14:0 | Myristic acid | | | | 13.1 |
| C 16:0 | Palmitic acid | 4.5 | 38 | 7.5 | 30.7 |
| C 16:0 at sn2 | Palmitic acid | | 21.7 | | |
| position# | | | | | |
| C 16:1 c | Palmitoleic | | | | 4.5 |
| | acid | | | | |
| C 18:0 | Stearic acid | 1.5 | 4.0 | 7.1 | |
| C 18:1 c | Oleic acid | 61.0 | 49.0 | 6.0 | 2.8 |
| C 18:2 cc | Linoleic acid | 19.8 | 7.0 | 8.0 | |
| C 18:3 ccc | α-linonenic | 8.0 | | 2.5 | |
| | acid (ALA) | | | | |
| C20:4n6 | Arachidonic | | | 46.2 | |
| | acid (ARA) | | | | |
| C 22:0 | Behenic acid | | | 3.0 | |
| C22:6n3 c | Docosahexaenoic | | | | 46.2 |
| | acid (DHA) | | | | |
| C 24:0 | Lignoceric | | | 11.3 | |
| | acid | | | | |

#expressed on total fatty acids
*fatty acid composition as analyzed according to AOCS method Cel-62. Fatty acids expressed in wt. % on total weigh of the fatty acid profile.
**fatty acid composition based on Osmond et al., Animals 2021, 11, 1185
***fatty acid composition of OPO oil commercially available

Step b) Subjecting the Blend Obtained from Step a) to a High-Shear Force

Prior to the loading, a blend of oils (according to table 1) was gently heated to 40° C. in a closed recipient for complete melting and subsequently slowly added to the isolated oleosomes are subjected to a high shear mixing using an IKA Ultra Turrax T25 Digital with a disperser S25N-8G. High shear mixing was applied for 3 minutes at a speed of 21500 rpm (which corresponds to a tip velocity of 6.7 m/s).

Table 3 specifies the oleosome composition of the isolated high-oleic (HO) sunflower oleosomes, the rapeseed oleosomes as well as the loaded isolated oleosomes according to Example 1.

TABLE 3
Composition of isolated high-oleic (HO) sunflower oleosomes and the
oleosome composition comprising the loaded isolated oleosomes.

| | Isolated HO | | | | | |
|---|---|---|---|---|---|---|
| | sunflower | | Loaded oleosomes | | | |
| | oleosomes | | Example 1 | | Example 2 | |
| Dry matter | 36.0 | wt. % | 64.0% | | 78.6% | |
| Protein content | 1.9 | wt. % | 0.6 | wt. % | 0.3 | wt. % |
| (expressed on | | | | | | |
| dry weight) | | | | | | |
| Total lipids content | 97.2 | wt. % | 99.1 | wt. % | 99.6 | wt. % |
| (expressed on | | | | | | |
| dry weight) | | | | | | |
| Free lipids (expressed | 3.5 | wt. % | n.a. | | n.a. | |
| on total weight | | | | | | |
| of lipids) | | | | | | |
| Lipids in oleosomes | 96.5 | wt. % | n.a. | | n.a. | |
| (expressed on total | | | | | | |
| weight of lipids) | | | | | | |

Fatty acid profile#

| C18.1 c | 81.3 | wt. % | 59.4 | wt. % | 53.9 | wt. % |
|---|---|---|---|---|---|---|
| C18.2 cc | 8.5 | wt. % | 10.0 | wt. % | 7.2 | wt. % |
| ALA | 0.2% | | 1.9 | wt. % | 0.2 | wt. % |
| DHA | — | | 0.9 | wt. % | — | |
| ARA | — | | 0.9 | wt. % | — | |
| C16:0 total | 4.3 | wt. % | 20.1 | wt. % | 33.0 | wt. % |
| C16:0 in sn2 position | n.a. | | 9.8 | wt. % | 21.7 | wt. % |
| (expressed on total | | | | | | |
| fatty acids) | | | | | | |

| | | | |
|---|---|---|---|
| n-6/n-3 | 42.5:1 | 3.9:1 | 36:1 |

n.a. not available

# The amount of fatty acids that is expressed in table 3 as wt. % of the fatty acid profile of the oleosome composition can also be expressed based on dry weight of the oleosome composition. For example, a loaded oleosome composition having C18.1 content of 59.4 wt. % based on the fatty acid profile of the oleosome composition, and the oleosome composition having a lipid content of 99.1 wt. % expressed on dry weight of the oleosome composition, has C18.1 content expressed on dry weight of the oleosome composition of 59.4×0.991=58.9 wt. %.

### Nutritional Compositions According to the Present Invention

Nutritional compositions according to the present invention have been prepared using the oleosome compositions from Example 1.

### Young Child Formula

Infant (young child) formulas for growing up milk were prepared using the oleosome composition from Example 1 (recipe A and recipe B). The recipe of the infant formula is disclosed in Table 4.

TABLE 4
Recipe of infant formula

| | Recipe A | Recipe B |
|---|---|---|
| Oleosome | 5.3 wt. % of | 2.8 wt. % of |
| composition ## | example 1 | example 2 |
| Additional | | Sunflower oil (1 wt. %) |
| lipids ## | | Flaxseed oil (0.067 wt. %) |
| | | Schizochytrium spec. |
| | | oil (0.05 wt. %) |
| | | Mortierella Alpina |
| | | oil (0.05 wt. %) |
| Skimmed milk | 26.1 wt. % | 26.1 wt. % |
| powder ## | | |
| Demineralized whey | 9.9 wt. % | 9.9 wt. % |
| protein ## | | |
| Lactose ## | 11.7 wt. % | 11.7 wt. % |
| Minerals, | QS (less than | QS (less than |
| vitamins ## | 0.5 wt. %) | 0.5 wt. %) |
| Water ## | to 100 wt. % | to 100 wt. % |

## amount of ingredients is expressed on the total weight of the infant formula

A stirring tank is filled with water at 20° C. to which skimmed milk powder, demineralized whey protein, and lactose were added and stirred to disperse for a period of time sufficient to hydrate. Afterwards, the minerals and vitamins are added and dispersed. Subsequently, the oleosome composition was added slowly while stirring to disperse well. The pH is adjusted to 6.7-7.0 if needed using disodium hydrogen phosphate or dipotassium hydrogen phosphate.

For recipe B specifically, additional the lipids (next to the lipids present in the loaded oleosomes), including lecithin, were added to the recipe in a next step. For that, the additional lipids, including lecithin, were preheated to 60° C. and subsequently added prior to a homogenization step in two stages. The mixture was homogenized in a first stage at a pressure of 150 to 200 bar, followed by a homogenization in a second stage at a pressure of 20 to 50 bar. The products obtained from recipe A and from recipe B were subsequently pasteurized at a temperature of 85° C. for 30 seconds before storing for later use.

Ready-to-feed infant formulae in liquid form are obtained. The composition of the infant formulae is shown in Table 5.

TABLE 5
Composition of the infant formula

| | | |
|---|---|---|
| Targeted range | Recipe A | Recipe B |

Based on the total weight of the infant formula

| | | | | | | |
|---|---|---|---|---|---|---|
| Proteins | 1.5 | wt. % | 1.5 | wt. % | 1.5 | wt. % |
| Carbohydrates | 7.0 | wt. % | 7.0 | wt. % | 7.0 | wt. % |
| Fats | 3.0-3.4 | wt. % | 3.35 | wt. % | 3.35 | wt. % |

Fatty acid profile (based on total weight of fats)

| C18.1 c | 30-70 | wt. % | 59.6% | | 44.5 | wt. % |
|---|---|---|---|---|---|---|
| C18.2 cc | 10-30 | wt. % | 25.2% | | 22.5 | wt. % |
| ALA | 1.0-2.0 | wt. % | 1.8% | | 1.4 | wt. % |
| DHA | 0.4-1.0 | wt. % | 0.9% | | 0.7 | wt. % |
| ARA | 0.4-1.0 | wt. % | 0.9 | wt. % | 0.7 | wt. % |
| C16:0 | 20-30 | wt. % | 21.4 | wt. % | 24.1 | wt. % |
| C16:0 at sn2 | 10-15 | wt. % | 10.4 | wt. % | 12.0 | wt. % |
| position | | | | | | |

Although certain aspects of the invention have been described, the scope of the appended claims is not intended to be limited solely to these specific aspects. The claims are to be construed literally, purposively, and/or to encompass equivalents. The scope of the present invention is defined by the appended claims. One or more of the objects of the invention are achieved by the appended claims.

1. An oleosome composition comprising loaded isolated vegetable oleosomes being isolated vegetable oleosomes loaded with one or more sources of triglycerides having saturated C16 fatty acids that are positioned at the sn2 position, which are present in the inside of said loaded isolated vegetable oleosomes and wherein at least 80 wt. % of the total weight of lipids in the composition is present in the oleosomes.

2. The oleosome composition according to claim 1, wherein the oleosomes are further loaded with one or more sources of long-chain polyunsaturated fatty acids (LC-PUFA) and/or with one or more sources of medium-chain fatty acids (MCFA).

3. The oleosome composition according to claim 1, wherein said isolated vegetable oleosomes are selected from the group consisting of isolated rapeseed oleosomes, isolated soybean oleosomes, isolated sunflower oleosomes, isolated linseed oleosomes, and a combination of two or more thereof.

4. The oleosome composition according to claim 1, comprising the one or more sources of triglycerides having saturated C16 fatty acids that are positioned at the sn2 position in an amount of from 0.5 to 90.0 wt. %, preferably from 1.0 to 80.0 wt. %, more preferably from 1.5 to 70 wt. % based on the total dry weight of the oleosome composition.

5. The oleosome composition according to claim 1, wherein the one or more sources of triglycerides having saturated C16 fatty acids that are positioned at the sn2 position has a fatty acid profile comprising from 30 to 50% by weight of saturated fatty acids having 16 carbon atoms and from 30 to 70% by weight of unsaturated fatty acids having 18 carbon atoms based on the total weight of the fatty acid profile of the one or more source, wherein 50 to 80%, such as from 52 to 75% by weight of the saturated fatty acids having 16 carbon atoms are present at the 2-position of a triglyceride.

6. The oleosome composition according to claim 1, comprising an amount of saturated C16 fatty acids that is positioned at the sn2 position of a triglyceride is in a range of from 0.075 to 36.0 wt. %, such as from 0.1 to 36.0 wt. %, preferably from 0.15 to 32 wt. %, such as 0.2 to 32.0 wt. %, more preferably 0.5 to 28.0 wt. % or from 0.234 to 26.25 wt. %, based on the total weight of the fatty acid profile of the oleosome composition.

7. The oleosome composition according to claim 1, further comprising lipophilic dietary bioactive substances, selected from the group consisting of oil-soluble vitamins, phytosterols, curcuminoids, carotenoids, and flavonoids, and combinations of two or more thereof, preferably wherein vitamin D is present as a lipophilic dietary bioactive substance; preferably wherein the content of vitamin D is in a range of from 0.1 to 1000 microgram per gram, based on the total weight of lipids of the oleosome composition.

8. The oleosome composition according to claim 1 wherein fatty acid profile of the lipids has a ratio of omega-6 fatty acids to omega-3 fatty acids (n-6:n-3) in a range of from 0.4 to 10.0, more preferably from 0.5 to 7.0, more preferably from 1.0 to 5.0.

9. The oleosome composition according to claim 1, wherein the oleosomes have an average globule diameter in a range of from 2.5 to 15 microns, preferably from 3.0 to 12 microns, more preferably from 4.0 to 10 microns.

10. A product comprising the oleosome composition according to claim 1, wherein said product is selected from the group consisting of food products, feed products, pharmaceutical products, personal care products, nutritional compositions and industrial products.

11. The product according to claim 10, being an infant formula, preferably growing-up milk.

12. The product according to claim 11, wherein the infant formula
has a total content of lipids in a range of from 2.5 to 4.5 wt. %, from 2.8 to 4.0 wt. %, or from 3.0 to 3.4 wt. % on total weight of the infant formula,
wherein at least 90%, at least 95%, or even substantially all of the lipids of the infant formula is present in the oleosome composition, and
wherein the oleosome composition
is comprising isolated vegetable oleosomes loaded with one or more source of triglycerides having saturated C16 fatty acids that are positioned at the sn2 position;
is comprising an amount of triglycerides having saturated C16 fatty acids that are positioned at the sn2 position of a triglyceride in a range of from 5.0 to 25.0 wt. %, from 8.0 to 20.0 wt. %, or from 10.0 to 18.0 wt. % based on the total weight of the fatty acid profile of the oleosome composition, and
wherein at least 80 wt. % of the total weight of lipids in the oleosome composition is present in the oleosomes.

13. A process for loading isolated vegetable oleosomes, wherein the process comprises the steps of:
a) blending one or more sources of triglycerides having saturated C16 fatty acids that are positioned at the sn2 position, and optionally other lipids with isolated vegetable oleosomes in a ratio of the source of triglycerides having saturated C16 fatty acids that are positioned at the sn2 position and optionally other lipids to oleosomes of from 1:99 to 95:5; and
b) subjecting the blend obtained from step a) to a high-shear force and obtaining an oleosome composition comprising isolated vegetable oleosomes loaded with one or more sources of triglycerides having saturated C16 fatty acids that are positioned at the sn2 position, wherein at least 80 wt. % of the total weight of lipids in the composition is present in the oleosomes.

14. The process according to claim 13, wherein the isolated oleosomes in step a) have a dry matter content in a range of from 30 to 80 weight % on the total weight of the oleosomes.

15. Use of the oleosome composition according to claim 1 as a carrier for triglycerides having saturated C16 fatty acids that are positioned at the sn2 position.

16. Use of the oleosome composition prepared according to the process of claim 13 as a carrier for triglycerides having saturated C16 fatty acids that are positioned at the sn2 position.

17. Use of the oleosome composition prepared according to the process of claim 14 as a carrier for triglycerides having saturated C16 fatty acids that are positioned at the sn2 position.
